(19) United States
(12) Patent Application Publication
(10) Pub. No.: US 2024/0140903 A1
(43) Pub. Date: May 2, 2024

(54) CHEMICAL ADDITIVES AND THEIR USE THEREOF FOR MODIFYING MECHANICAL PROPERTIES OF PVC AND PREVENTING THE FORMATION OF SURFACE DEFECTS DURING PVC CALENDERING

(71) Applicant: THE ROYAL INSTITUTION FOR THE ADVANCEMENT OF LEARNING / MCGILL UNIVERSITY, Montréal (CA)

(72) Inventors: Richard LEASK, Montréal (CA); Roya JAMARANI, Gloucester (CA); Omar Garcia VALDEZ, Kingston (CA); Jim A. NICELL, Kirkland (CA); Milan MARIC, Montréal (CA); Matthew HALLORAN, Montréal (CA); Kushal PANCHAL, Brampton (CA); Roozbeh MAFI, Kitchener (CA)

(21) Appl. No.: 18/257,695

(22) PCT Filed: Dec. 16, 2021
(86) PCT No.: PCT/CA2021/051815

§ 371 Date: Jun. 15, 2023

Related U.S. Application Data
(60) Provisional application No. 63/192,738, filed on May 25, 2021.
(60) Provisional application No. 63/126,937, filed on Dec. 17, 2020.

(51) Int. Cl. C07C 69/675; C08J 5/18; C08K 5/11

(52) U.S. Cl. CPC C07C 69/675; C08J 5/18; C08K 5/11; C08J 2327/06

(57) ABSTRACT

The disclosure relates to a compound of formula wherein Y, X, Ra, Rb, R₁ and R₂ are defined herein, PVC compositions comprising the compounds disclosed herein, as well as methods for modulating properties of a PVC, including reducing or preventing the formation of observable gas checks in calendered films, or other mechanical properties such as tensile strength and/or glass transition temperature.

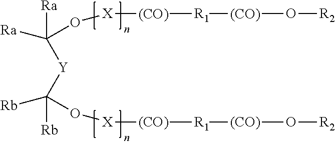

### BACKGROUND OF THE DISCLOSURE

Many plastic formulations require additive compounds to be blended to modify the mechanical properties of the final product and aid in polymer processing. It is common that up to 40% of a plastic product weight is comprised of compounds added to increase flexibility and elongational properties and improve processing characteristics. We have developed a series of new compounds that when used as additives to PVC formulations can prevent surface defects created during polymer film processing (calendering). These compounds have also been shown to reduce the glass transition temperature and increase the elongation at break of PVC blends, making them more flexible. These compounds demonstrate improved permanence (lower leaching) than comparative additives such as diisononyl phthalate (DINP).

Calendering is a process used to make plastic sheets which involves passing melts through heated rolls (i.e., the calender) to produce a continuous film with controllable thickness. Surface defects are common in calendered PVC films and their frequency increases with film thickness. A ‘gas check’ is a common defect believed to be created by entrapped gas escaping the surface in the calender bank. Gas checks (herein referred as) have also been described as specks, gas entrapments, air bubbles, air inclusions, or flecking in the polymer literature. Gas checks reduce overall film quality, resulting in inferior products that are often rejected and discarded during the manufacturing process. As a low percentage additive (8-10%), our compounds have been shown to completely prevent gas check surface defects in calendering PVC films.

It should be noted that gas checks not only reduce the quality of film produced but can also degrade the mechanical integrity of the film. Currently, industrial production of PVC films without gas checks requires operator experience and trial and error tuning of the processing parameters such as the speed of calendering, distance and temperature of the rolls. This process tuning puts limits on the material properties that can be achieved. To our knowledge, there exist no other additives to prevent the formation of gas check defects.

At high percentages (15-40%) additives are often used to “plasticize” PVC formulations, reducing the stiffness of the final product and improving flexibility. Over the past decades, the ubiquity of plasticizer compounds in our environment and their know toxicity have become of great concern and compounds such as phthalates have been subjected to regulatory bans in some applications, leading to a search for alternatives.

The present inventors have found that the addition of the compounds disclosed herein in PVC, provides for the modulation of properties, including reducing or preventing the formation of observable gas checks in calendered films, or other mechanical properties such as tensile strength and/or glass transition temperature.

### SUMMARY OF THE DISCLOSURE

An aspect of the disclosure relates to a compound of formula

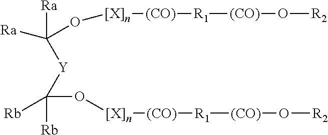

wherein Y, X, Ra, Rb, R₁ and R₂ are defined herein.

In one aspect, there is provided a method for reducing the number of observed gas checks in a calendered PVC film, comprising forming and calendering a composition comprising a compound as defined herein and a PVC polymer.

In one aspect, there is provided a method for modulating at least one property of a PVC comprising adding a compound as defined herein to a PVC polymer.

In a further aspect, there is provided a PVC composition or product comprising PVC and a compound as defined herein.

A process for forming a PVC product comprising compounding a PVC polymer and a compound as defined herein, and forming said PVC polymer and compound as defined herein into a shaped PVC product.

### BRIEF DESCRIPTION OF THE DRAWINGS

FIG. 1 is a bar graph representing the number of gas checks per m² of calendered film with reference compounds and compounds as defined herein;

FIG. 2. is a bar graph representing the liquid viscosities of compounds as defined herein;

FIG. 3 illustrates the dimensions of tensile bars used in tensile tests;

FIG. 4 illustrates the elongation at break for PVC blends with compounds as defined herein and a reference compound;

FIG. 5 shows the tensile data for calendered films produced with compounds as defined herein compared to a reference compound;

FIG. 6 shows the observed glass transition temperatures of PVC blends made with compounds as defined herein; and

FIG. 7 shows the leaching of compounds from PVC blends into hexane.

### DETAILED DISCLOSURE

In one aspect, there is provided a compound of formula

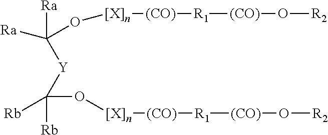

wherein Y is

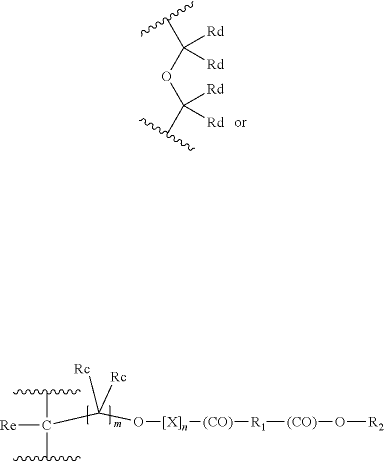

X is —(CO)—(CH₂)₅—O—
Ra, Rb, Rc, Rd and Re are each independently H, or a lower straight or branched alkyl or lower cycloalkyl;
R₁ is an alkylene or an alkenylene chain;
R₂ is an alkylene chain;
m is 0 or 1;
each n is an integer independently selected from 0-3.

In one embodiment, the compounds have the formula:

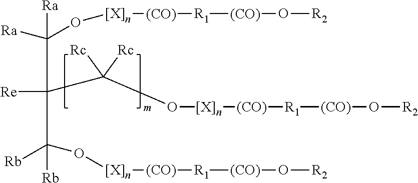

wherein X, Ra, Rb, Rc, Re, R₁, R₂ m and n are as defined herein.

In a further embodiment, the compounds have the formula:

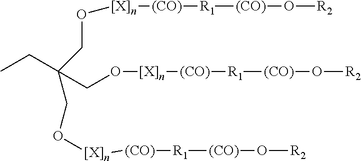

wherein X, R₁, R₂ and n are as defined herein.

In a further embodiment, the compounds have the formula:

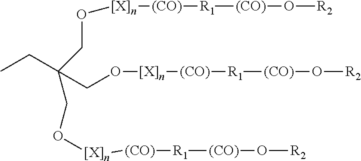

wherein X, R₁, R₂ and n are as defined herein.

In still a further embodiment, the compounds have the formula

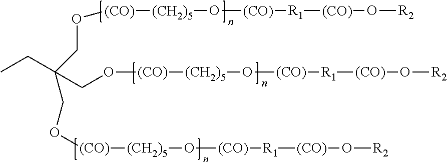

wherein R₁, R₂ and n are as defined herein.

In still a further embodiment, the compounds have the formula

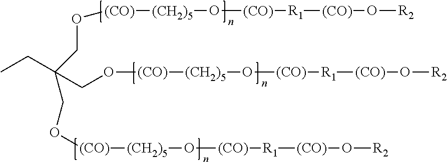

wherein R₁, R₂ and n are as defined herein.

In one embodiment, any of the residue

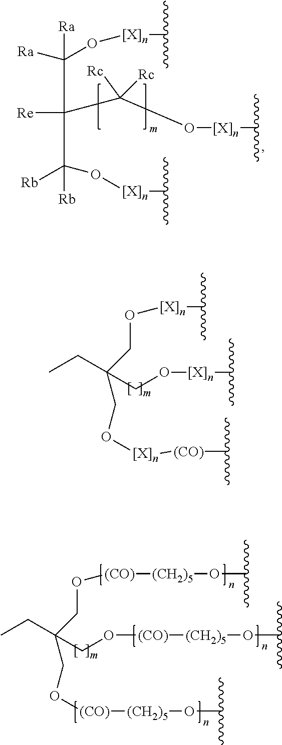

has an integer average Mn value of from about 300 to about 900.

In one embodiment, Y is

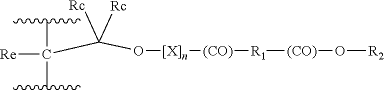

In one embodiment, Y is

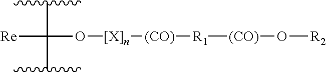

In one embodiment, R₁ is C2-C4 alkylene or C2 alkenylene.

In one embodiment, R₂ is C2-C12 alkyl, preferably C4 to C10 alkyl chain.

In one embodiment, Ra, Rb, Rc, Rd and Re are each independently H, or a C1-C6 linear and branched alkyl.

In one embodiment, Ra, Rb, Rc, Rd and Re are each independently H, a methyl or ethyl.

In one embodiment, Ra, Rb, and Rc, or Ra, Rb, and Rd are each H.

In one embodiment, Re is H, a methyl or ethyl.

In one embodiment, the amount of compound as defined herein, for example used for calendering a PVC blend, is at least about 8 pHr, or at least about 10 pHr.

In one embodiment, the viscosity of a compound disclosed herein is at least about 300 cP at 25° C.

The term “alkyl”, as used herein, is understood as referring to a saturated, monovalent unbranched or branched hydrocarbon chain which can also be optionally substituted when valencies allow. Examples of alkyl groups include, but are not limited to, C1-12 alkyl groups, provided that branched alkyls comprise at least 3 carbon atoms, such as C3-10. Lower straight alkyl may have 1 to 6 or preferably 1 to 3 carbon atoms; whereas branched lower alkyl comprise C3-6.

The term “alkylene”, as used herein, is understood as referring to a bivalent alkyl residue, wherein alkyl is as previously defined, which can further be optionally substituted when valencies allow, and including without limitation —(CH₂)₂—, —(CH₂)₃—, —(CH₂)₄—.

The terms “alkenyl” represent optionally substituted linear or branched hydrocarbon moiety which has one or more double bonds, preferably one, in the chain. The number of carbon atoms can be the same as those in “alkyl” provided that there is at least 2 carbon atoms. An “alkenyl” may have 2 to 6, preferably have 2 to 4 carbon atoms.

The term “alkenylene”, as used herein, is understood as referring to a bivalent alkenyl residue, wherein alkenyl is as previously defined, and including without limitation —(CH═CH)—, —(CH═CH)—(CH═CH)—, —(CH═CH)—(CH₂)₂— and —(CH₂)₂—(CH═CH).

### EXAMPLES

### Chemicals and Reagents

PCL triol (Mₙ=900) (99%), PCL diol (Mₙ=530) (99%), fumaric acid (99%), oxalic acid (98%), adipic acid (99%), 1-butanol (99%), 1-decanol (98%), n-heptanol (99%), and succinic acid (99%) were purchased from Sigma Aldrich, Missouri, USA. Poly(ε-caprolactone) (PCL) triol (Mₙ=300, 540) (99%) was purchased from Scientific Polymer Products Inc., NY, USA. Sulfuric acid (96%), hexanes (99%), and stearic acid were purchased from Fisher Scientific, New Hampshire, USA. Diheptyl succinate (DHPS) was synthesized in accordance with the method previously described in B. M. Elsiwi et al., ACS Sustainable Chem. Eng., (2020) 8 (33), 12409-12418. Diisononyl phthalate (DINP) (99.8%), PVC resin (70K suspension), antimony oxide Hi-Tint (99.68%), silica (99%), stearic acid (99%), barium/zinc stabilizer (1.046 specific gravity at 20° C.), and acrylic processing aid (99.8%) were supplied by Canadian General-Tower Limited (CGT Ltd.). All chemicals and reagents were used as received without further purification.

### General Synthesis of PCL-Based Additives

Synthesis of the star-shaped PCL analogs was performed via a two-step reaction sequence in a single round-bottom flask. PCL triol (1 equiv.) was massed directly into a 1 L round-bottom flask followed by the addition of the appropriate diacid (3 equiv.). Benzene (250 mL) was subsequently added to the flask, and the mixture was stirred at room temperature for 5 minutes. Catalytic amounts of sulfuric acid (0.15 equiv.) were then added dropwise to the reaction mixture. The flask was fitted with a Dean-Stark apparatus and a condenser, then submerged in a pre-heated oil bath at 100° C. After two hours, the mixture was removed from the oil bath and was allowed to reach room temperature. The appropriate alcohol (3 equiv.) was then added to the same flask equipped with the Dean-Stark apparatus and condenser, and the mixture was submerged in an oil bath set to 100° C. After two hours, the flask was cooled to room temperature and concentrated to afford the star-shaped PCL analogs as viscous oils. The code names for the PCL analogs are listed in Table 1.

### 1. PCL₃₀₀-Succ-C7:

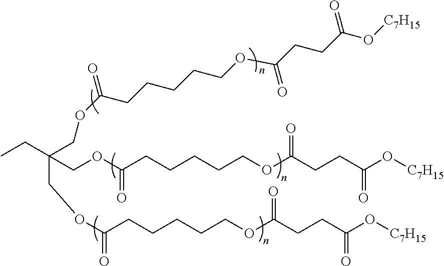

The compound was prepared according to the general procedure described above using 169.5 g (0.565 mol, 1 equiv.) of PCL-triol (Mₙ=300), 200.2 g (1.695 mol, 3 equiv.) of succinic acid, 196.9 g (1.695 mol, 3 equiv.) of n-heptanol, and 4.7 mL (0.088 mol, 0.15 equiv.) of H₂SO₄ to afford 495.5 g (0.554 mol) of PCL-300-C7 as a viscous light orange oil in a 98% yield.

ii. PCL₅₄₀-Succ-C7:

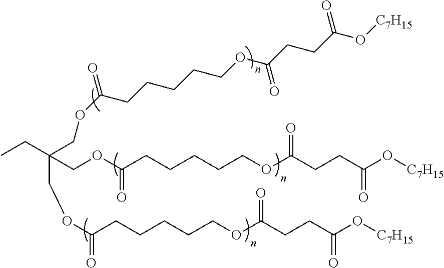

The compound was prepared according to the general procedure described above using 240.0 g (0.444 mol, 1 equiv.) of PCL-triol (Mₙ=540), 157.5 g (1.333 mol, 3 equiv.) of succinic acid, 154.9 g (1.333 mol, 3 equiv.) of n-heptanol, and 3.7 mL (0.069 mol, 0.15 equiv.) of H₂SO₄ to afford 489.3 g (0.431 mol) of PCL-540-C7 as a viscous brown oil in a 97% yield.

IR (neat) v=2929.81, 2858.36, 1731.23, 1152.17 cm⁻¹.

iii. PCL₉₀₀-Succ-C7:

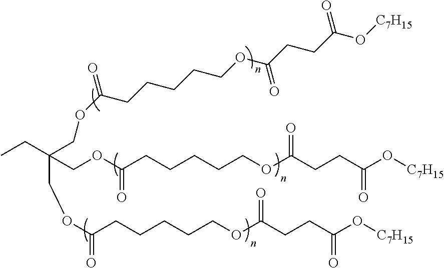

The compound was prepared according to the general procedure described above using 310.0 g (0.344 mol, 1 equiv.) of PCL-triol (Mₙ=900), 122.0 g (1.033 mol, 3 equiv.) of succinic acid, 120.1 g (1.033 mol, 3 equiv.) of n-heptanol, and 2.8 mL (0.054 mol, 0.15 equiv.) of H₂SO₄ to afford 499.4 g (0.334 mol) of PCL-900-C7 as a viscous yellow oil in a 97% yield.

iv. PCL₅₄₀-Succ-C10:

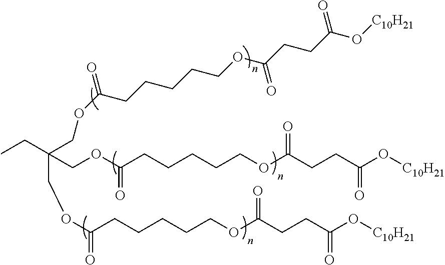

The compound was prepared according to the general procedure described above using 86.40 g (0.160 mol, 1 equiv.) of PCL-triol (Mₙ=540), 56.68 g (0.480 mol, 3 equiv.) of succinic acid, 75.97 g (0.480 mol, 3 equiv.) of 1-decanol, and 1.27 mL (0.024 mol, 0.15 equiv.) of H₂SO₄ to afford 195.7 g (0.155 mol) of PCL-540-C10 as a viscous brown oil in a 97% yield.

v. PCL₅₄₀-Succ-C4:

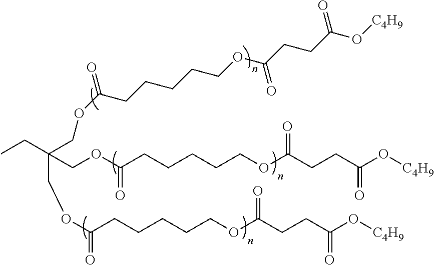

The compound was prepared according to the general procedure described above using 108 g (0.200 mol, 1 equiv.) of PCL-triol (Mₙ=540), 70.85 g (0.600 mol, 3 equiv.) of succinic acid, 1.6 mL (0.030 mol, 0.15 equiv.) of H₂SO₄ and 44.47 g (0.600 mol, 3 equiv.) of 1-butanol to afford 199.7 g (0.198 mol) of PCL-540-C4 as a viscous brown oil in a 99% yield.

vi. PCL₅₄₀-Fum-C7:

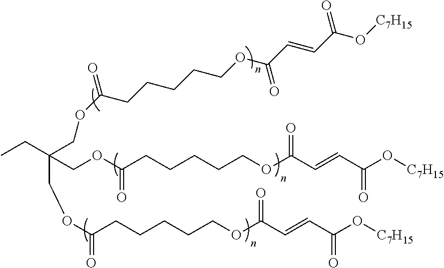

The compound was prepared according to the general procedure described above using 99.90 g (0.185 mol, 1 equiv.) of PCL-triol (Mₙ=540), 64.40 g (0.555 mol, 3 equiv.) of fumaric acid, 64.49 g (0.555 mol, 3 equiv.) of n-heptanol, and 1.48 mL (0.027 mol, 0.15 equiv.) of H₂SO₄ to afford 204.6 g (0.181 mol) of PCL-Fumarate-C7 as a viscous brown oil in a 98% yield.

vii. PCL₅₄₀-Adi-C7:

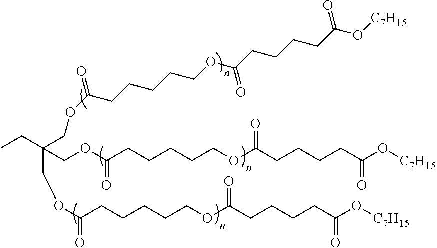

The compound was prepared according to the general procedure described above using 91.80 g (0.170 mol, 1 equiv.) of PCL-triol (Mₙ=540), 74.53 g (0.510 mol, 3 equiv.) of adipic acid, 59.26 g (0.510 mol, 3 equiv.) of n-heptanol, and 1.36 mL (0.026 mol, 0.15 equiv.) of H₂SO₄ to afford 198.9 g (0.163 mol) of PCL-Adipate-C7 as a viscous brown oil in a 96% yield.

viii. PCL₅₄₀-Oxa-C7:

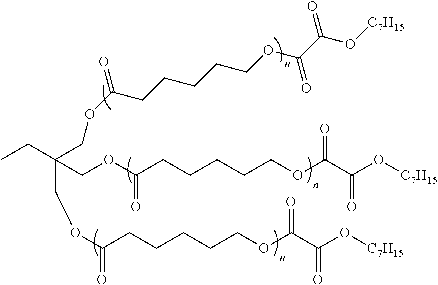

The compound was prepared according to the general procedure described above using 114.38 g (0.212 mol, 1 equiv.) of PCL-triol (Mₙ=540), 57.21 g (0.635 mol, 3 equiv.) of oxalic acid, 73.83 g (0.635 mol, 3 equiv.) of n-heptanol, and 1.69 mL (0.032 mol, 0.15 equiv.) of H₂SO₄ to afford 216.1 g (0.206 mol) of PCL-Oxalate-C7 as a viscous brown oil in a 97% yield.

ix. Linear-PCL530-Succ-C7

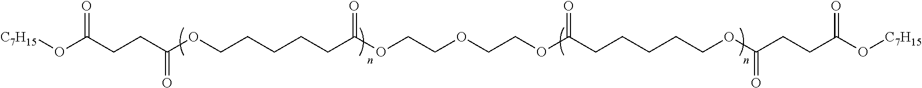

The compound was prepared according to the general procedure described above using 114.48 g (0.216 mol, 1 equiv.) of PCL-diol (Mₙ=530), 51.01 g (0.432 mol, 3 equiv.) of succinic acid, 50.2 g (0.432 mol, 3 equiv.) of n-heptanol, and 1.7 mL (0.032 mol, 0.15 equiv.) of H₂SO₄ to afford 190.1 g (0.205 mol) of Linear PCL-C7 as a viscous orange oil in a 95% yield.

### Alternate Synthesis of PCL-Based Additives

Diheptyl succinate (DHPS) was synthesized in a one-step, solvent-free reaction, as previously reported by Elsiwi, B. M., et al. (ACS Sustain. Chem. Eng., (2020)). Tributylsuccinate-terminated poly(caprolactone) (PCL₅₄₀-Succ-C4) was synthesized using the same method described previously by Jamarani, R., et al. (Polym. Eng. Sci., (2021)). The remainder of the star-shaped PCL analogs were synthesized using a two-step reaction method similar to a previously described method by Jamarani, R., et al. (supra), but modified to remove any use of the benzene solvent, and subsequent solvent removal steps through rotary evaporation. In a first step, PCL triol (one stoichiometric equivalent) and the diacid reagent (three equiv.) were added to a three-necked round bottom flask fitted with a Dean-Stark trap and a condenser. The mixture was then stirred at room temperature for 5 min. A catalytic amount of sulfuric acid (0.15 equiv.) was added to the reaction mixture and the mixture was heated to 110° C. and stirred continuously. Once at temperature, nitrogen gas was bubbled through the mixture for 90 min to promote the removal of water. The mixture was then cooled to room temperature. The alcohol reagent (three equiv.) was then added directly to the flask and the mixture was re-heated to 110° C., at which point nitrogen gas was again bubbled through the mixture for 90 min. The mixture was then cooled to room temperature. The resulting viscous oils were not further purified. Table 1 shows the reagents used to synthesize each plasticizer as well as the abbreviated plasticizer names that will be used hereinafter. The linear PCL analog was synthesized using the same procedure as the star-shaped PCLs except for the use of two stoichiometric equivalents of diacid and alcohol reagents, respectively, instead of the three equivalents used for the star-shaped molecules. The resulting linear-PCL₅₃₀-Succ-C7 was obtained as a viscous oil and was not further purified.

The purpose for this alternate method was for optimizing the synthesis of the PCL-based additives by avoiding the use of organic solvent with the goal of developing a set of conditions that reduced reaction waste and were amenable to large-scale production. The inventors were able to demonstrate the successful incorporation of the heptyl-succinate groups onto the PCL core of PCL₅₄₀-Succ-C7, as observed through ¹H NMR, using our modified one-pot method that was based on the solvent-free method previously described by Elsiwi et al. (supra).

TABLE 1

| PCL-based | | | # of PCL | Diacid | | Mn (g |
|---|---|---|---|---|---|---|
| additive name | Code | PCL core | branches | reagent | Alcohol | mol⁻¹) |
| Poly(caprolactone) | PCL₅₄₀-triol | PCL-triol (Mn = 540) | 3 | | | 540 |
| triol | | | | | | |
| Triacetate- | PCL₅₄₀-Acet | PCL-triol (Mn = 540) | 3 | | | 666 |
| terminated | | | | | | |
| poly(caprolactone) | | | | | | |
| Triheptylsuccinate- | PCL₃₀₀-Succ-C7 | PCL-triol (Mₙ = 300) | 3 | Succinic | Heptanol | 897 |
| terminated | PCL₅₄₀-Succ-C7 | PCL-triol (Mₙ = 540) | 3 | acid | | 1137 |
| poly(caprolactone) | PCL₉₀₀-Succ-C7 | PCL-triol (Mₙ = 900) | 3 | | | 1497 |
| Tributylsuccinate- | PCL₅₄₀-Succ-C4 | PCL-triol (Mₙ = 540) | 3 | Succinic | Butanol | 1009 |
| terminated | | | | acid | | |
| poly(caprolactone) | | | | | | |
| Tridecylsuccinate- | PCL₅₄₀-Succ-C10 | PCL-triol (Mₙ = 540) | 3 | Succinic | Decanol | 1261 |
| terminated | | | | acid | | |
| poly(caprolactone) | | | | | | |
| Triheptyloxalate- | PCL₅₄₀-Oxa-C7 | PCL-triol (Mₙ = 540) | 3 | Oxalic | Heptanol | 813 |
| terminated | | | | acid | | |
| poly(caprolactone) | | | | | | |
| Trihepylfumarate- | PCL₅₄₀-Fum-C7 | PCL-triol (Mₙ = 540) | 3 | Fumaric | Heptanol | 891 |
| terminated | | | | acid | | |
| poly(caprolactone) | | | | | | |
| Triheptyladipate- | PCL₅₄₀-Adi-C7 | PCL-triol (Mₙ = 540) | 3 | Adipic | Heptanol | 981 |
| terminated | | | | acid | | |
| poly(caprolactone) | | | | | | |
| Diheptylsuccinate- | Linear-PCL₅₃₀- | PCL-diol (Mₙ = 530) | 1 | Succinic | | 928 |
| terminated | Succ-C7 | | | acid | | |
| poly(caprolactone) | | | | | | |

### Film Production

A total mass of 300 g of the formulations in Table 2 were manually premixed in a bowl. The blend was then premixed using a Hartek two-roll mill HTR-300 (d=120 mm, T=160° C., 45 rpm) for 7 minutes. The milled film was fed into a lab-scale calender (d=180 mm, T=160-170° C., P=45 psi hps, 50 rpm). A film gauge of 0.4 mm+/−0.05 mm was produced by adjusting the calender nip distance. The process was repeated 3 to 4 times for each blend. Plasticized PVC films were prepared to a final concentration of 55 phr (32.5 wt %) plasticizer.

TABLE 2
Blend Formulations

| DINP 55 | DINP 65 | 4 phr | 8 phr | 10 phr | 55 phr |
|---|---|---|---|---|---|
| 55 phr | 65 phr | 55 phr | 55 phr | 55 phr | — |
| DINP | DINP | DINP | DINP | DINP | |
| — | — | 4 phr gas | 8 phr gas | 10 phr gas | 55 phr gas |
| | | check | check | check | check |
| | | additive* | additive* | additive* | additive* |

Every blend contains:
100 phr PVC 70K suspension resin, 7 phr antimony oxide, 1 phr silica, 1 phr stearic acid, 4 phr barium/zinc stabilizer (2-aminonicotinic acid), and 1 phr acrylic processing aid for flexible vinyl (2-Propenoic acid, 2-methyl-, methyl ester, polymer with ethyl 2-propenoate).
*represents the compounds that were tested for their ability to prevent the formation of gas checks.
DINP was used in all blends other than those in which the gas check additives were added at 55 phr.

Table 3 discloses the structures of certain known or previously reported chemicals discussed herein.

TABLE 3

| | |
|---|---|
| DINP | 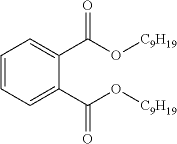 |
| DHPS | 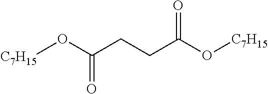 |
| PCL-Triol | 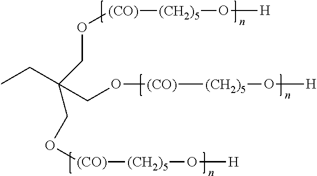 |
| PCL-Acet | 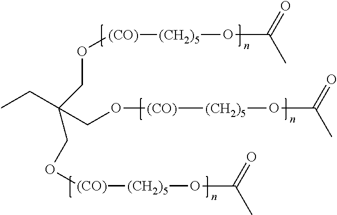 |

### Counting Gas Checks

Gas checks were manually counted without magnification using a 7 cm×7 cm grid in 3 regions of each film. The average number of gas checks per film was normalized per m² of film.

Table 4 summarizes the results from the repeated films produced for each formulation.

TABLE 4

| | Concen- | | Number of | Average number of gas |
|---|---|---|---|---|
| Additive | tration | | blends | checks per m² |
| DINP | 55 | phr | 6 | 5115 |
| DINP | 65 | phr | 1 | 5680 |
| PCL₅₄₀-triol | 55 | phr | 1 | 9864 |
| PCL₅₄₀-Acet | 55 | phr | 1 | 6122 |
| DHPS | 55 | phr | 1 | 6241 |
| PCL₅₄₀-Succ-C7 | 55 | phr | 3 | 0 |
| | 10 | phr | 3 | 204 |
| | 8 | phr | 3 | 879 |
| | 4 | phr | 3 | 4807 |
| PCL₃₀₀-Succ-C7 | 55 | phr | 3 | 0 |
| | 10 | phr | 3 | 0 |
| | 8 | phr | 3 | 0 |
| | 4 | phr | 3 | 5193 |
| PCL₉₀₀-Succ-C7 | 55 | phr | 3 | 0 |
| | 10 | phr | 3 | 0 |
| | 8 | phr | 3 | 227 |
| | 4 | phr | 3 | 5941 |
| PCL₅₄₀-Fum-C7 | 55 | phr | 1 | 0 |
| PCL₅₄₀-Oxa-C7 | 55 | phr | 1 | 0 |
| PCL₅₄₀-Adi-C7 | 55 | phr | 1 | 0 |
| PCL₅₄₀-Succ-C4 | 55 | phr | 1 | 0 |
| PCL₅₄₀-Succ-C10 | 55 | phr | 1 | 0 |
| Linear-PCL₅₃₀-Succ-C7 | 55 | phr | 1 | 0 |
| PCL-glycerol analog | 55 | phr | 1 | 1480 |

### Rheology

Liquid additive viscosities were measured by steady-shear tests using a strain-controlled rheometer (Anton Paar MCR 302, Anton Paar Canada, St-Laurent, Quebec, Canada) with parallel plate geometry (25 mm plate diameter) with a CTD 540 convection oven and double gap geometry for low-viscosity samples. Shear rate was increased logarithmically from 0.1 s⁻¹ to 100 s⁻¹ at 25° C. (±0.3° C.).

### Preventing Gas Check Formation Using Additives

It was found that using PCL₅₄₀-Succ-C7 in PVC blends at 55 phr resulted in the production of calendered films with no gas checks. In contrast, the use of DINP, which is the current industrial plasticizing standard, in PVC blends at 55 phr resulted in films with an average of 5115 gas checks per m² of film

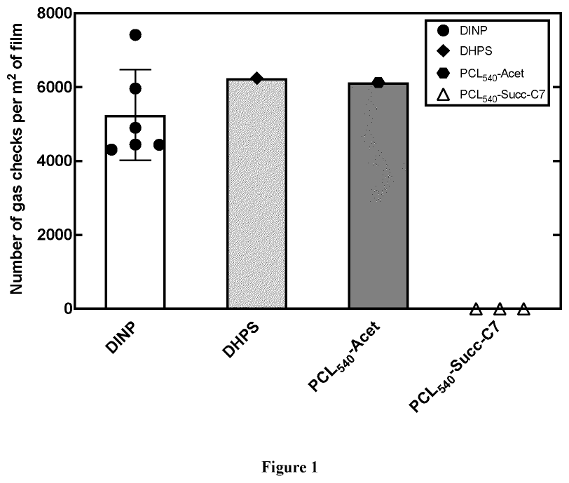

It can be seen in FIG. 1 that DHPS, another previously reported compound, was not able to prevent the formation of gas checks when blended with PVC at 55 phr, with an average of 6241 gas checks per m² of film, showing no decrease compared to DINP.

PCL₅₄₀-triol was found to be incompatible with PVC which was evidenced by several observations including: (i) significantly delayed film formation rate on the mill compared to other blends; (ii) extremely poor quality of the final film, which was very brittle, with many cracks and holes, and exhibited similar physical properties to unplasticized PVC; (iii) the material coming off the mill was covered in a thick oily layer, further suggesting the immiscibility of PCL₅₄₀-triol with PVC.

PCL₅₄₀-Acet, another previously known compound, did not remove gas checks, producing films with an average of 6122 gas checks per m² of film (FIG. 1).

Our additive, PCL₅₄₀-Succ-C7, was blended at increasing concentrations from 4 phr to 55 phr. All blends (except for the 55 phr PCL₅₄₀-Succ-C7 blend) contained 55 phr DINP as a primary plasticizer with PCL₅₄₀-Succ-C7 added at concentrations of 4 phr, 8 phr, and 10 phr. At 8 phr, there was a significant reduction in the number of gas checks compared to the DINP control and 4 phr films, with an average of 877 gas checks per m². There were virtually no gas checks in the films with over 10 phr additive.

A film with 65 phr DINP was also calendered for comparison. It was found that the 65 phr DINP film still contained an average of 5680 gas checks per m² of film.

We found that both PCL₃₀₀-Succ-C7 and PCL₉₀₀-Succ-C7, two additives with different molecular weights, prevented the formation of gas checks in calendered films completely at 55 phr with no difference in effectiveness at 4 phr, 8 phr, and 10 phr compared to PCL₅₄₀-Succ-C7. Therefore, we conclude that at values between 300 and 900, the molecular weight of the PCL-triol oligomer core had no effect on gas check removal during calendering.

PCL₅₄₀-Fum-C7, PCL₅₄₀-Oxa-C7 and PCL₅₄₀-Adi-C7, which contain different acid groups, completely removed gas checks at 55 phr, as shown in Table 4. Therefore, between the analogs tested, the type of diacid did not influence their activity.

PCL₅₄₀-Succ-C4 and PCL₅₄₀-Succ-C10, with different alkyl chain lengths, both removed all gas checks at 55 phr and behaved similarly to PCL₅₄₀-Succ-C7 (see Table 4). Therefore, we conclude that within this range, alcohol chain length did not influence the prevention of gas check formation.

The final structural element that was investigated was the role of branching in the additive molecule. All of the aforementioned structures, including the original PCL₅₄₀-Succ-C7 have a 3-armed star shaped structure. We synthesized a new linear molecule from a PCL-diol core, keeping the succinate diacid and heptanol end groups constant. We observed that the -PCL₅₃₀-Succ-C7 removed all gas checks at 55 phr, similarly to the star-shaped molecules, suggesting that branching was not the reason for gas check removal (see Table4).

### Viscosity

We investigated the correlation of the viscosity of the additives disclosed herein and the avoidance of the formation of gas check defects.

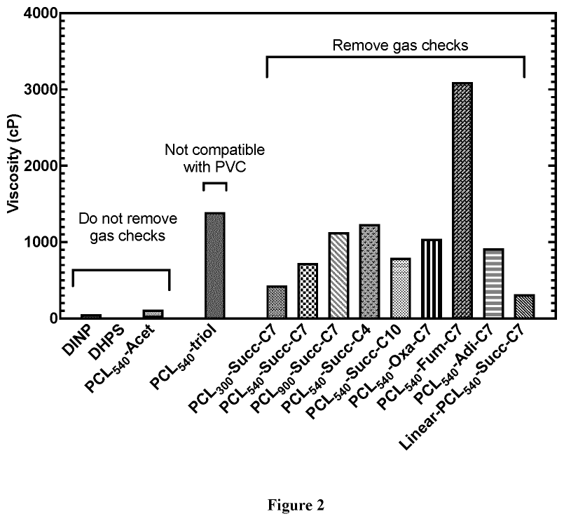

The viscosities of the additives disclosed herein were measured at 25° C., shown in FIG. 2. It can be seen that the viscosities of the additives that remove gas checks when blended with PVC are higher than those of the additives that do not remove gas checks. DINP, DHPS, and PCL₅₄₀-Acet (which do not remove gas checks) have viscosities that range from approximately 10 cP to 115 cP. The viscosities of the PCL-based additives (which all remove gas checks) range from approximately 315 cP for Linear-PCL₅₃₀-Succ-C7 to 3100 cP for PCL₅₄₀-Fum-C7.

This trend suggests that viscosity plays a role in the additives' ability to prevent gas checks from forming. It is worth noting that PCL₅₄₀-triol, which was found to be incompatible with PVC, has a high viscosity. Despite having a viscosity within the ‘favorable’ range, it is not a suitable additive to remove gas checks since it does not blend well with PVC and drastically reduces film quality, making the resulting product unusable.

### Materials for Extrusion of Bulk Samples

Suspension PVC resin (UPVC; K50) was provided by Solvay Benvic, Chevigny-Saint-Sauveur, France. Epoxidized soybean oil was purchased from Chemtura Corporation (Philadelphia, PA, USA) as a thermal stabilizer for PVC, and stearic acid was purchased from Fisher Scientific (Montreal, QC, Canada) as a lubricant.

### Extrusion of PVC-Plasticizer Blends

Plasticized blends were prepared to final concentrations of 20 phr (parts per hundred resin, 16.67 wt %), 40 phr (28.57 wt %) and 60 phr (37.50 wt %) using a conical intermeshing twin-screw extruder (Haake Minilab, Thermo Electron Corporation, Beverly, MA, USA) containing a screw diameter of 5/14 mm conical, a screw length of 109.5 mm, and a batch size of 3 g. The extruder was operated at 140 ° C. throughout using a screw rotation speed of 30 min⁻¹. Blends were prepared using the following three-step sequence.

Initially, UPVC was combined with 20 phr plasticizer, 4 phr epoxidized soybean oil, and 5 phr stearic acid and fed into the extruder. The resulting extrudate was manually cut into small fragments, and then recycled through the extruder. In the second step, another 20 phr plasticizer was added and extruded to achieve a total concentration of 40 phr plasticizer. The resulting blend was again recycled through the extruder. In the final step, another 20 phr plasticizer was added (to the 40 phr blend) and extruded to achieve a final concentration of 60 phr. The resulting blend was again recycled through the extruder and the extrudate was manually cut into pellets.

### Preparation of Tensile Bars of Bulk Specimens

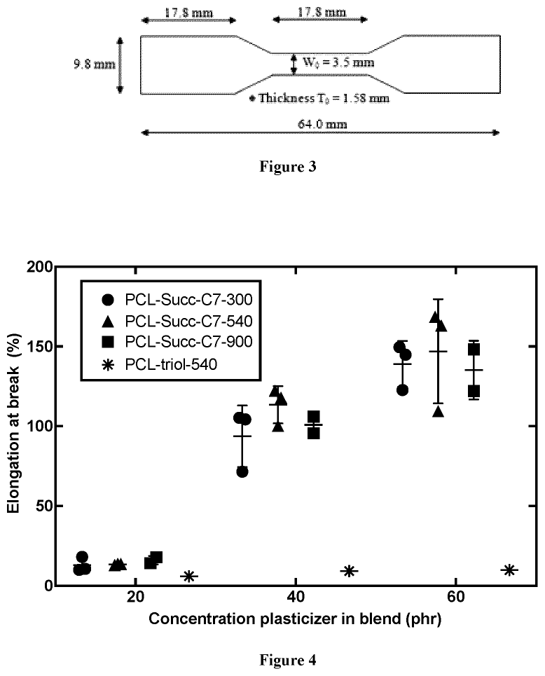

The plasticized PVC was pressed into tensile bars, dimensions shown in FIG. 3 (Erythropel, H. C.; Maric, M.; Cooper, D. G., Chemosphere 2012, 86 (8), 759-766) using a hot press (Carver Manual Hydraulic Press with Watlow Temperature Controllers, Carver Inc., Wabash, IN, USA) and a corresponding mold. The extrudate of the plasticized PVC blend used to fill the tensile bar mold. Pressing was performed at 165° C. and 1 MPa for 10 min before degassing, then at 2 MPa for 10 min, and finally at 3 MPa for 30 min and then cooled to room temperature at which point the pressure was released. After cooling, the disks were removed from the mold and placed in desiccator (Drierite, Fisher Scientific, Montreal, QC, Canada) for a minimum of 48 h before tensile testing.

### Tensile Testing of Bulk Specimens

All tensile testing was performed using the Yamazu Easy Test tensile tester with a load cell of 500 N after the test bars had spent at least 48 h in the desiccator. The exact dimensions (thickness and width) of the test bar were measured using electronic calipers (Electronic Outside Micrometer, Fowler Tools & Instruments) and recorded after which the test bars were clamped into the apparatus and were exposed to a strain rate of 5 mm min⁻¹. Both the elongation distance and force on the test bar were automatically recorded by the attached computer until break of the test bar. Using this data, a stress-strain curve was generated. The elongation at break, which is the strain at the breaking point of test. bar was obtained. All reported data is the average of 3 samples. The procedure was adapted from the ASTM standard for tensile testing (ASTM D-638, 2003). Results are presented in FIG. 4. It can be seen that the three different molecular weight PCL₃₀₀-Succ-C7, PCL₅₄₀-Succ-C7 and PCL₉₀₀-Succ-C7 synthesized, demonstrated an increase in elongation at break with concentration when blended with PVC, whereas PCL-triol (Mₙ=540) alone did not.

### Tensile Testing of Film Specimens

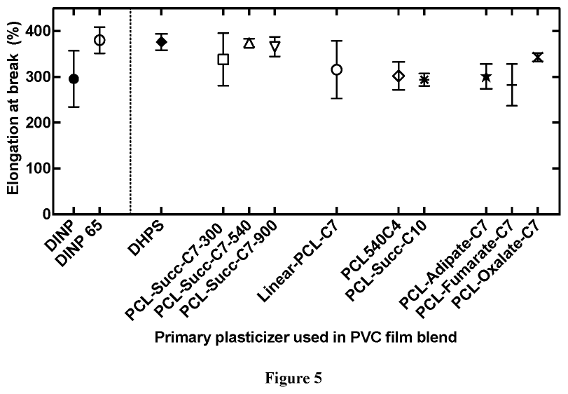

All tensile testing was performed using an Instron Tensile Tester 3365 equipped with a Bluehill Universal 5 kN load cell following the ASTM D882 protocol. Test bars were punched from films into dimensions of 1×6″, and a testing speed of 20″/min with an initial gap of 2″ was used throughout. Results are presented in FIG. 5. It can be seen that the additives disclosed herein, in addition to removing film surface defects also improved film elongation of the calendered films as disclosed and prepared in the examples above.

### Glass Transition Temperature

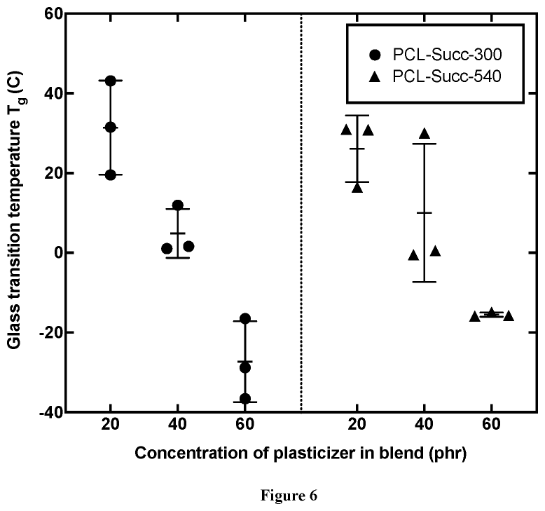

Glass transition temperature was measured by Differential Scanning Calorimettry (DSC) using the protocol described in B. M. Elsiwi et al., ACS Sustainable Chem. Eng., (2020) 8 (33), 12409-12418. The glass transition temperature of plasticized PVC blends was measured using a TA Instruments Q2000 differential scanning calorimeter. A previously-established temperature-modulated differential scanning calorimetry (MDSC) protocol was used (Erythropel, H. C., M. Maric, and D. G. Cooper, Chemosphere, 86, 8 (2012)). Briefly, between 5-10 mg of sample was weighed and loaded into a Tzero Hermetic aluminum pan then into the DSC sample holder. The MDSC protocol comprised two cool-heat cycles. In the first cycle, the sample was cooled to −90° C. and held isothermally for 5 min. The cooled sample was then exposed to a linear heating ramp of 2° C. min⁻¹ with a superimposed modulated heating (amplitude=1.27° C., period=60 s) until it reached 100° C. and was held isothermally for 5 min. This cycle was repeated a second time. DSC results were analyzed using TA Universal Analysis software (V4.5A). Glass transition temperature was determined from the reversible heat flow curve of the second heating cycle using the T_{g} tool. FIG. 6 shows that, in line with the other observed mechanical properties, there is a decrease in T_{g} with increasing concentration of the added compounds.

### Leaching Tests

Leaching tests were performed for 6 hours at 50° C. in 200 mL of hexane. Samples were stored in a dessicator and weighed before each test and then dried in under vacuum at 35° C. for 7 days and then weighed to obtain the final mass.

The discs that were used for each leaching test were prepared by heat pressing 8 calendered films at 165° C. for 1 minute under 5 tonnes of force and 4 minutes under 20 tonnes of force. The samples were cooled under pressure using circulating cold water.

In other tests, the disks that were used for the leaching tests were prepared from the previously calendered PVC films (55 phr plasticizer). A circular punch (d=25 mm) was used to cut film samples that were layered into stacks of eight and placed in a circular mold. A heat press (Carver Manual Hydraulic Press with Watlow Temperature Controllers) was used to press the films at 165° C. for 1 min under 5 tons of force and 4 min under 20 tons of force. The samples were cooled under pressure using circulating cold water. They were then removed from the mold and placed in a desiccator (Drierite, Fisher Scientific) for a minimum of one week before the leaching tests.

Leaching tests were performed using a protocol adapted from ASTM D1239. Each disk was weighed prior to the start of the test then suspended in a 250 mL Erlenmeyer flask using an aluminum wire. The flasks were filled with 200 mL of hexanes, stoppered, and set in a shaker at 100 rpm and 50° C. for 6 h. At the end of this time, the disks were removed from the flasks and dried under vacuum at 35° C. for 7 days and then weighed. The percent weight loss of the plasticizer was calculated using the following equation:

$$Plasticizer loss = \frac{m - {m}_{o}}{0.325 \times m} \times 100$$

where m represents the final mass of the disk after the leaching test and m₀ represents the initial mass of the disk before the leaching test. Since the concentration of plasticizer is known to be 55 phr, or 32.5 wt %, of the PVC blend, the initial mass of plasticizer was calculated by multiplying the mass of the disk by 0.325. Three separate leaching tests were performed for each plasticizer and the results shown are presented as the mean and standard deviation of the three tests.

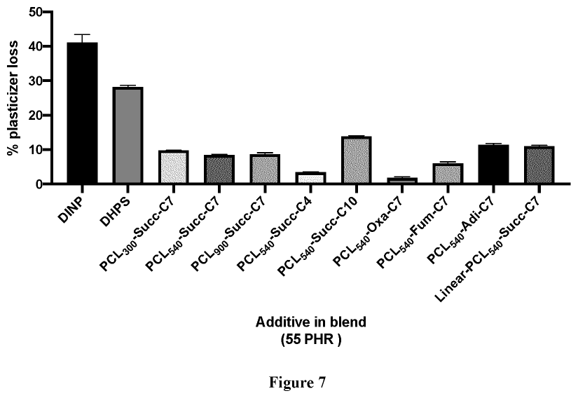

FIG. 7 shows that the additives disclosed herein, in addition to removing film surface defects, also reduced leaching into hexane. All additives disclosed herein had leaching values between 2% and 14% while DINP and DHPS had leaching values of 41% and 28%.

### Effect of Molecular Weight

There was no difference in the amount of plasticizer leached between PCL₃₀₀-Succ-C7, PCL₅₄₀-Succ-C7 and PCL₉₀₀-Succ-C7, with all three plasticizers exhibiting leaching between 8%-10%, all significantly lower than DINP and DHPS. The three plasticizers are comprised of a PCL-triol core (of increasing molecular weights of 300, 540, and 900 g/mol), a succinic acid linker and a 7-carbon alkyl cap (see FIG. 1 C). However, despite their differences in molecular weight, these three analogs displayed similar leaching behavior.

### Effect of Alkyl Chain Length

A significant effect of alkyl chain length on migration was found when comparing PCL540-Succ-C4, PCL540-Succ-C7, and PCL540-Succ-C10. We observed a trend of increasing leaching (3%, 8%, and 14%) with increasing alkyl chain length from four to ten carbons. All three additives contained an identical PCL-triol core, with Mn of 540 g/mol, and a succinic acid linker, however they were synthesized using alcohols of increasing chain lengths (i.e., butanol, heptanol, decanol). The increase in leaching with alkyl chain length is likely the result of a decrease in the relative proportion of polar groups on the plasticizer, which are thought to provide strong points of interaction with the polymer through the formation of solvating dipoles on the PVC chain Thus, having longer non-polar aliphatic functional groups on the plasticizer means that fewer points of attraction exist between polymer and plasticizer, resulting in increased migration.

### Effect of Acid Type

Similarly, a significant effect of the dicarboxylic acid on migration resistance was observed, with increasing leaching with increasing length of aliphatic group within the acid. All four structures, PCL540-Oxa-C7, PCL540-Succ-C7, PCL540-Fum-C7, and PCL540-Adi-C7, are comprised of two ester functional groups, with differing carbon chain lengths between the esters. PCL540-Oxa-C7, which is made from oxalic acid, the smallest dicarboxylic acid, is comprised of two adjoining esters with no aliphatic group between them and exhibited the lowest leaching of the four plasticizers at 2%. Conversely, PCL540-Adi-C7, made from adipic acid, which contains two carboxylate groups separated by four methylene groups, has the longest aliphatic linker of the four plasticizers, and exhibited the highest leaching at 11%. There was no statistical difference in the amount of plasticizer leached between PCL540-Succ-C7 and PCL540-Fum-C7, which demonstrated 6-8% leaching, and both contain two linking carbons. Succinic acid, which contains a C—C single bond is simply the saturated analog of fumaric acid which contains a C═C double bond. The use of different acids modifies the ratio of polar to non-polar groups in each plasticizer, with an increase in the ratio of non-polar groups (i.e., longer aliphatic chains) corresponding to higher levels of leaching, which is consistent with the trend observed for the different alkyl capping groups.

### Effect of Branching

Finally, the effect of branching on migration resistance was investigated using Linear-PCL₅₃₀-Succ-C7. All of the other PCL-based additives in this study were synthesized from a PCL-triol core, resulting in a three-armed star structure, while Linear-PCL₅₃₀-Succ-C7 was synthesized from a PCL-diol, resulting in a linear structure. Leaching of the linear analog was compared to PCL₅₄₀-Succ-C7 and PCL₃₀₀-Succ-C7 since all three additives were synthesized from an oligomeric PCL core, succinic acid, and heptanol, and have similar molecular weights. We found no significant difference between the leaching of Linear-PCL₅₃₀-Succ-C7 and the two branched analogs, with all three additives leaching between 8-11%. These findings indicate that there is no correlation between the degree of branching of PCL-based plasticizers on leaching.

### Additional Compounds—Glycerol-PCL Compounds

A new series of compounds were prepared. These new compounds are PCL-glycerol based analogs such as

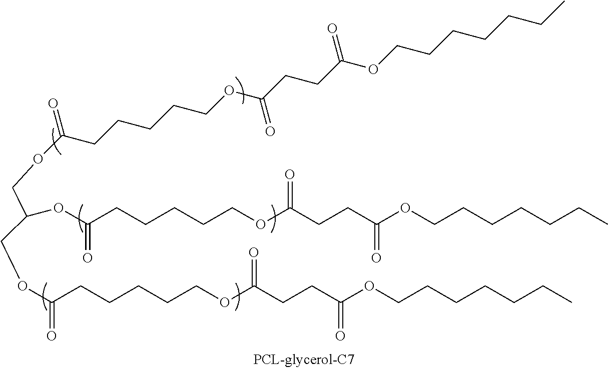

Table 5 summarizes the results obtained with the PCL-Glycerol compounds tested

TABLE 5
Samples were measured in accordance to ASTM D882
in the machine direction (MD). Sample size is
n = 8 with an average thickness of 15.5 mm.

| | 100% Modulus (lb_{f}) | Tensile at break (lb_{f}) | Elongation (%) |
|---|---|---|---|
| Mean | 23.32 | 42.28 | 322.7 |
| Max | 24.6 | 46.08 | 363.37 |
| Min | 21.99 | 38.53 | 295.37 |

Further, Table 6 reports the average gas check per sq.m.

TABLE 6
Average number of gas check per area based
on the method of calculation adopted herein

| | | | | R1 | R2 | R3 | Average | | |
|---|---|---|---|---|---|---|---|---|---|
| | | | | per | per | per | (n per | | |
| | R1 | R2 | R3 | area | area | area | sq. m) | Counting area basis | |
| F1 | 0 | 3 | 10 | 0 | 612 | 2041 | 884 | | |
| F2 | 0 | 10 | 11 | 0 | 2041 | 2245 | 1429 | | |
| F3 | 1 | 5 | 18 | 204 | 1020 | 3673 | 1633 | L (m) | 7.00E−02 |
| F4 | 0 | 8 | 21 | 0 | 1633 | 4286 | 1973 | W (m) | 7.00E−02 |

| | | | |
|---|---|---|---|
| Average gas | 1480 | Area | 4.90E−03 |
| check per sq. m | | | |

1. A compound of formula 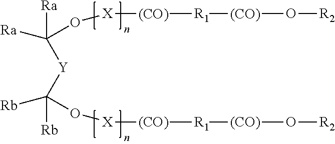
wherein Y is 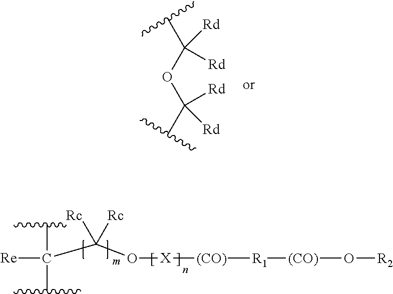
X is —(CO)—(CH₂)₅—O—
Ra, Rb, Rc, Rd and Re are each independently H, or a lower straight or branched alkyl or lower cycloalkyl;
R₁ is an alkylene or an alkenylene chain;
R₂ is an alkylene chain;
m is 0 or 1;
each n is an integer independently selected from 0-3.

2. The compound of claim 1, wherein:
Y is 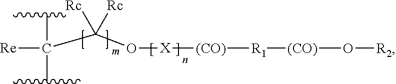
Ra, Rb, Rc, Re, X, R₁, R₂, m, and n are as defined in claim 1.

3. The compound of claim 1, wherein:
Y is 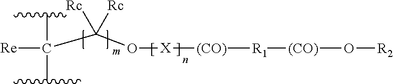
X is —(CO)—(CH₂)₅—O—;
Ra, Rb, and Rc are each hydrogen;
R₁ is an alkylene or an alkenylene chain;
R₂ is an alkylene chain;
m is 1;
each n is an integer independently selected from 0-3; and
Re is ethyl.

4. The compound of claim 1, wherein:
Y is 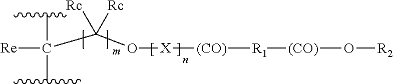
X is —(CO)—(CH₂)₅—O—;
Ra, Rb, and Rc are each hydrogen;
R₁ is an alkylene or an alkenylene chain;
R₂ is an alkylene chain;
m is 1;
each n is an integer independently selected from 0-3; and
Re is ethyl.

5. The compound of claim 1, wherein R1 is a C2-C4 alkylene or a C2 alkenylene.

6. The compound of claim 1, wherein R2 is a C2-C12 alkyl.

7. The compound of claim 1, wherein R2 is a C4-C10 alkyl.

8. The compound of claim 1, wherein Ra, Rb, Rc, Rd, and Re are each independently H or a C1-C6 linear or branched alkyl.

9. The compound of claim 1, wherein Ra, Rb, Rc, Rd, and Re are each independently H, a methyl or an ethyl.

10. The compound of claim 1, wherein Ra, Rb, and Rc, or Ra, Rb and Rd are each H.

11. The compound of claim 1 wherein Re is H, methyl or ethyl.

12. A method for reducing the number of observed gas checks in a calendered PVC film, comprising forming and calendering a composition comprising a compound as defined in claim 1 and a PVC polymer.

13. A method for modulating at least one property of a PVC comprising adding a compound as defined in claim 1 to a PVC polymer.

14. A PVC composition or product comprising PVC and a compound as defined in claim 1.

15. A process for forming a PVC product comprising compounding a PVC polymer and a compound as defined in claim 1, and forming said PVC product into a shaped PVC product.
